(19) United States
(12) Patent Application Publication
(10) Pub. No.: US 2011/0174348 A1
(43) Pub. Date: Jul. 21, 2011

(54) AUTOMATED HAND-WASHING DEVICE

(75) Inventor: Mauri Helenius, Helsinki (FI)

(73) Assignee: HANDYSEPT OY, Helsinki (FI)

(21) Appl. No.: 13/060,330

(22) PCT Filed: Aug. 31, 2009
(86) PCT No.: PCT/FI2009/000079

§ 371 Date: Mar. 31, 2011

(30) Foreign Application Priority Data
Sep. 3, 2008 (FI) 20080495

(51) Int. Cl. B08B 3/00

(52) U.S. Cl. 134/56 R

(57) ABSTRACT

An automated hand-washing device includes a case-like housing having two openings (A), into which the user can introduce the hands for disinfection and automated spraying of an antispetic solution operating without hand contact through spray nozzles, from where the antispetic solution jet is directed precisely to the correct spot; the fingertips, the fingers and part of the palm. The openings (A) are oval, vertically elongated ellipses and they contain substantially equally shaped recesses, which urge the hands into the correct position for the period of disinfection.

This invention relates to an automated hand-washing device, which consists of a case-like housing having two openings, into which the user can introduce the hands for disinfection, and comprising automatic spraying of antiseptic solution operating without hand contact through spray nozzles, from which the antiseptic solution jet is directed precisely to the right spot; the fingertips, the fingers and part of the palm. The purpose of use of the device is hand disinfection, which enhances hand hygiene and consequently prevents bacterial contagion.

Previously known solutions of this type have been disclosed e.g. by the following references:
U.S. Pat. No. 5,924,148
EP1211306
EP1233749
EP1241968
EP1171568
   in addition to hand dispensers and spray bottles known per se.

These prior art solutions have the following drawbacks:
the dispensed amount is inexact, the substance is dispensed onto the hand as a single dose, in a sort of lump, so that an even disinfection result cannot be ensured,
most devices require hand contact,
high consumption of antiseptic solution relative to the result obtained,
too slow to be apt for use by large publics,
placed in sanitary facilities.

The purpose of this invention is to overcome the drawbacks mentioned above. The automated hand-washing device of the invention is characterised by the following features: the openings in the device are shaped as oval, vertically elongated ellipses, and the openings contain substantially equally shaped recesses, which urge the hands into the correct position for the period of disinfection.

Various embodiments of the invention are defined in the dependent claims of the set of claims.

The automated device of the invention has the following advantages:
it is fully automated,
the spray jet is directed to exactly the right spot; the fingertips, the fingers and part of the palm,
the user is unable to impede the end result by his/her own actions
optimated dose,
rapid function, the entire process being completed in less than a second, making the device apt for use by the large public,
the design of the device allows installation in restaurants and on any premises where people dwell, work or circulate,
the feedback of the speech syntheziser encourages especially children to enhanced hand hygiene.

The invention will be explained below by means of an example and with reference to the accompanying drawings, in which

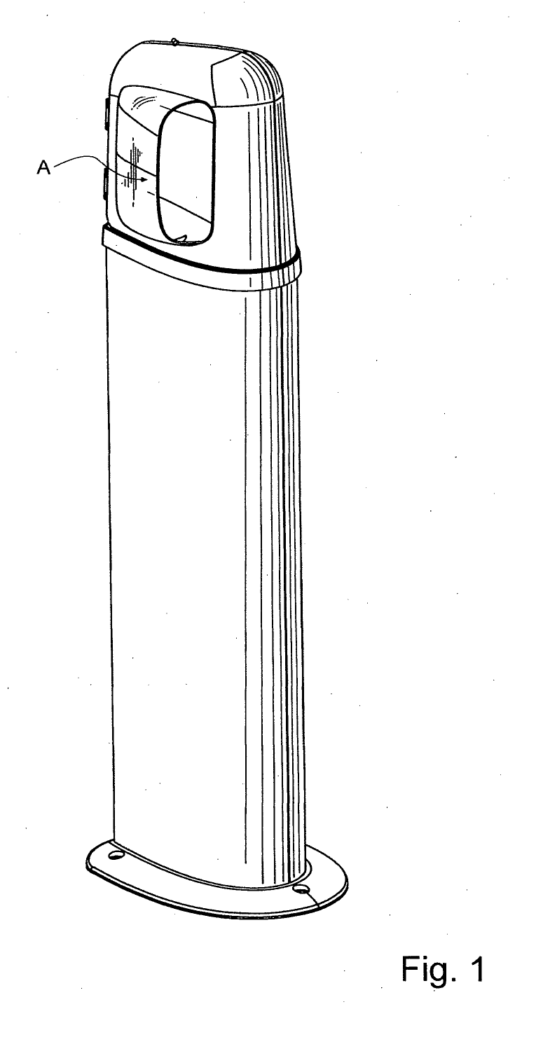

FIG. 1 illustrates a floor model of the automated hand-washing device,

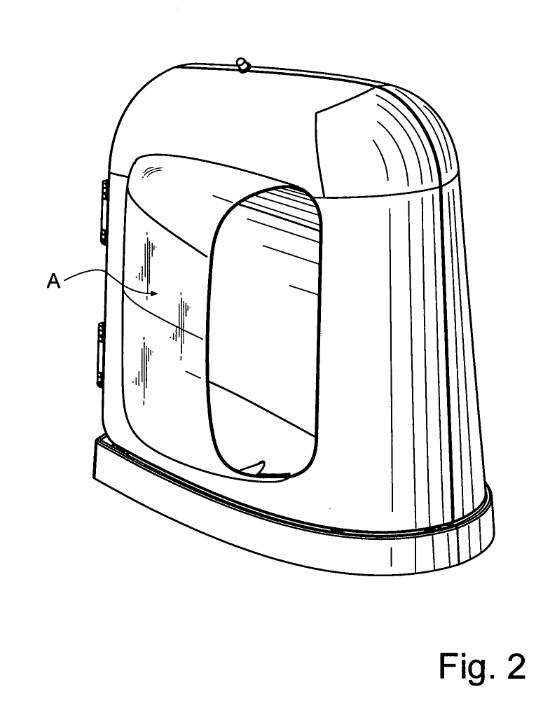

FIG. 2 illustrates a table model of the automated hand-washing device,

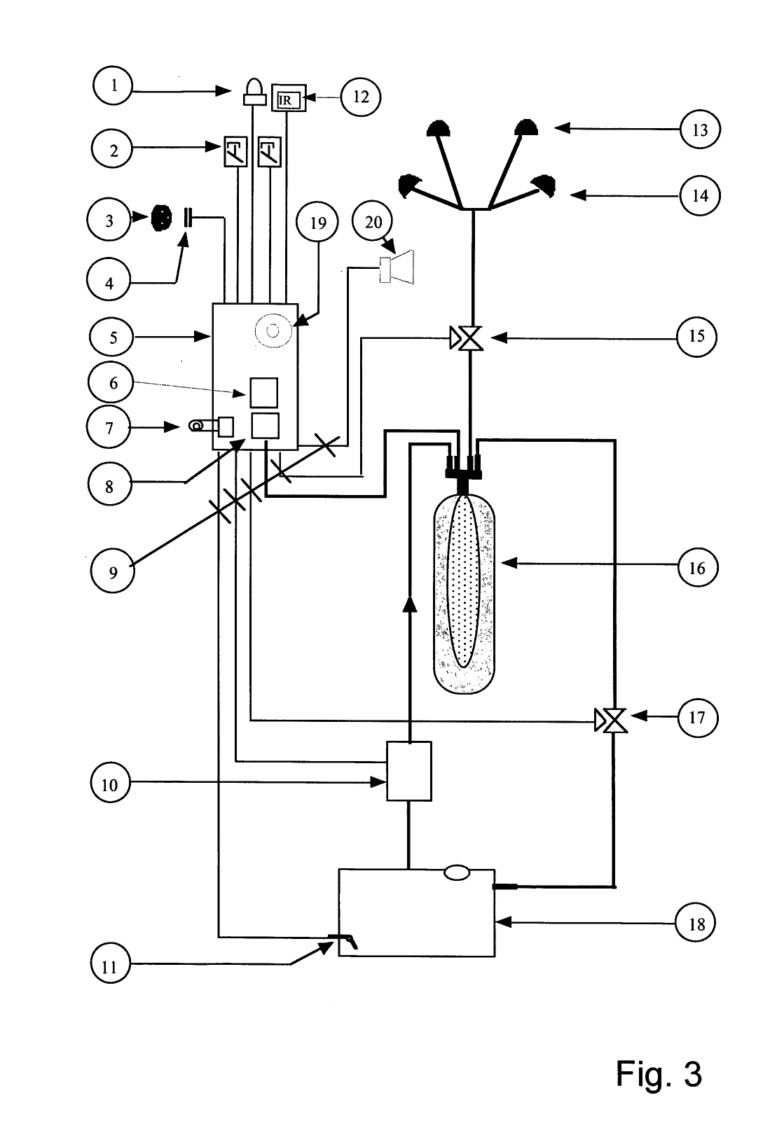

FIG. 3 is a schematic view of the automated hand-washing device.

The automated hand-washing device consists of a case-like housing having two openings A, into which the user can introduce the hands for disinfection, and comprises automatically functioning, touchless spraying of antiseptic solution through spray nozzles 13, 14, from which the antiseptic solution jet is directed exactly to the right spot; the fingertips, the fingers and part of the palm. The openings are shaped as oval, vertically elongated ellipses, and they contain substantially equally shaped recesses, which urge the user's hands into the right position for the period of disinfection. The recesses are tapered inwardly. The fluid nozzles 13, 14 are located within the recesses. The automated device comprises a pressure accumulator 16, to which antiseptic solution is pumped by the fluid pump 10 and further through the solenoid valve 15 to the nozzles 13, 14. The pressure accumulator 16 consists of a pressure tank, which contains a flexible membrane bag, the operating pressure being 4-6 bars and the energy being stored in the pressure accumulator 16. The automated device also includes a processor, which monitors the spraying by means of the solenoid valve 15. The operating voltage of the device is 12 V DC. The external parts of the device are replacable, such as the outer walls of the openings, the “lugs”, which allow for a custom-designed appearance.

The automated device may additionally be equipped with a speech synthetizer, in which user-specific data and instructions have been stored. The motion detector included in the device activates friendly instructions stored in the speech synthetizer, which encourage the user to approach for disinfection of his/her hands.

The disinfecting operation is activated when the user has put his/her hands into the openings A on the sides of the device and the sensors 2 have sensed them. The sensors 2 give a control to the processor on the circuit board 5. The processor gives a preset control command to the solenoid valve 15, which then opens. After this, the fluid stored under overpressure in the pressure accumulator 16 passes to the nozzles 13, 14 and is dispersed on the hands, which are thus disinfected.

The software of the processor reacts on pressure variations by reading the pressure sensor 8 located on the circuit board. As the pressure drops to the lower limit, the processor activates the fluid pump 10, which transfers fluid from the tank 18 to the pressure accumulator 16. The processor stops the pump 10 when the set upper pressure limit has been achieved.

When the fluid in the fluid tank 18 has sunk to the lower limit, the sensor 11 informs the processor that the tank should be filled. A lower limit alarm causes a light diode 1 to be lit and generates a tone in an acoustic alarm 19.

When the fluid tank 18 is to be filled, the left-hand side of the housing is opened, whereby the door switch 4 informs the processor. The processor then switches off all the functions, allowing the device to be handled without spraying antiseptic solution as the device is touched.

The tank 18 is filled and the “door” is closed. The door switch then sends a signal about the access door being closed. The “closed” data from the door switch 4 activates the pump 10 and opens the solenoid valve 17, so that the fluid starts circulating in an internal circuit within the device. Any air present within the system is then conducted to the fluid tank 18.

The solenoid valve 17 is closed at the end of a pre-set period. The pump 10 goes on operating and transferring fluid to the pressure accumulator 16 until the upper pressure limit is reached. Then the pump 10 is stopped and the device is ready for operation.

The device may also be equipped with a motion detector 12. As the detector has detected a human who passes by without using the device, an invitation to hand washing stored in the speech synthetizer is heard over the loudspeaker 20.

The device is not restricted to the example given above, but may vary within the scope of the following claims. Thus the device may come in other models than the floor model and the table model described above. The current source may be e.g. a 230 V/12 V DC transformer or a chargeable accumulator.

List of parts of the automated hand-washing device

| pos. | Number | units | Item |
|---|---|---|---|
| | 1. | 1 | light diode |
| | 2. | 2 | capacitive sensor |
| | 3. | 1 | door switch magnet |
| | 4. | 1 | door switch |
| | 5. | 1 | circuit board |
| | 6. | 1 | speech synthetizer |
| | 7. | 1 | voltage feed 12 VDC |
| | 8. | 1 | pressure switch |
| | 9. | 4 | cabling |
| | 10. | 1 | fluid pump |
| | 11. | 1 | lower limit alarm sensor |
| | 12. | 1 | motion detector |
| | 13. | 2 | fluid nozzle |
| | 14. | 2 | fluid nozzle |
| | 15. | 1 | solenoid valve |
| | 16. | 1 | pressure accumulator |
| | 17. | 1 | solenoid valve |
| | 18. | 1 | antiseptic solution tank |
| | 19. | 1 | acoustic alarm |
| | 20. | 1 | loudspeaker |

1. Automated hand-washing device, consisting of a case-like housing having two openings (A), into which the user's hands are introduced for disinfection and comprising automated spraying of antiseptic solution operating without hand contact through spray nozzles (13, 14), from which the antispetic solution jet is directed precisely to the correct spot: the fingertips, the fingers and part of the palm, characterised in that the openings (A) are oval, vertically elongated ellipses containing substantially equally shaped recesses, which urge the hands into the correct position for the period of disinfection.

2. Automated hand-washing device as defined in claim 1, characterised in that the recesses are tapered inwardly.

3. Automated hand-washing device as defined in claim 1, characterised in that one or more nozzles (13, 14) are provided within the recess.

4. Automated hand-washing device as defined in claim 1, characterised in that the automated device comprises a pressure accumulator (16), to which antiseptic solution is pumped by a fluid pump (10), and from there onwards through a solenoid valve (15) to nozzles (13, 14).

5. Automated hand-washing device as defined in claim 1, characterised in that the pressure accumulator (16) consists of a pressure tank comprising a flexible membrane bag, the operating pressure being 4-6 bars and energy being stored in the pressure accumulator (16).

6. Automated hand-washing device as defined in claim 1, characterised in that the automated device comprises a processor for monitoring the spraying process by means of a solenoid valve (15).

7. Automated hand-washing device as defined in claim 1, characterised in that the operating voltage of the automated device is 12 V DC.

8. Automated hand-washing device as defined in claim 1, characterised in that the external parts of the automated device are replacable, such as the outer walls of the openings, the “lugs”, which allow for a customer-designed appearance.

9. Automated hand-washing device as defined in claim 1, characterised in that the automated device is provided with a speech synthetizer, in which user-oriented information and instructions have been recorded.

10. Automated hand-washing device as defined in claim 9, characterised in that the automated device is equipped with a motion detector, which starts instructions and/or encouragements recorded in the speech synthetizer for passers-by to approach in order to disinfect their hands.

11. Automated hand-washing device as defined in claim 1, characterised in that the automated device is equipped with a venting circuit between the pressure accumulator (16) and the fluid tank (18) for conducting any air present in the system to the fluid tank.

12. Automated hand-washing device as defined in claim 2, characterised in that the automated device comprises a pressure accumulator (16), to which antiseptic solution is pumped by a fluid pump (10), and from there onwards through a solenoid valve (15) to nozzles (13, 14).

13. Automated hand-washing device as defined in claim 3, characterised in that the automated device comprises a pressure accumulator (16), to which antiseptic solution is pumped by a fluid pump (10), and from there onwards through a solenoid valve (15) to nozzles (13, 14).
